(19) United States
(12) Patent Application Publication
(10) Pub. No.: US 2017/0325967 A1
(43) Pub. Date: Nov. 16, 2017

(54) FULLY EXPANDABLE INTERVERTEBRAL FUSION IMPLANT

(71) Applicant: FACET-LINK INC., Rockaway, NJ (US)

(72) Inventors: Helmut D. LINK, Hamburg (DE); Klaus DMUSCHEWSKY, Hamburg (DE)

(73) Assignee: FACET-LINK INC., Rockaway, NJ (US)

(21) Appl. No.: 15/523,300

(22) PCT Filed: Oct. 30, 2015
(86) PCT No.: PCT/EP2015/075265

§ 371 Date: Apr. 28, 2017

(30) Foreign Application Priority Data
Oct. 31, 2014 (EP) 14191306.1

(51) Int. Cl. A61F 2/44; A61F 2/46; A61F 2/30

(52) U.S. Cl. CPC A61F 2/4455; A61F 2/4611; A61F 2002/30556; A61F 2002/30537; A61F 2002/30405; A61F 2002/30176; A61F 2002/30156; A61F 2002/30579; A61F 2002/30525

(57) ABSTRACT

An intervertebral fusion implant for the fusion of two adjacent vertebrae includes an elongate, adjustable support body, of which the lower and upper cover surfaces are designed to bear on end-plates of the adjacent vertebrae, wherein a side bracket is provided, which is pivotable laterally about a hinge, and of which the bottom face and top face are designed to bear on the end-plates. The support body furthermore is provided, on the cover surface thereof, with a lifting plate which, by means of a lifting mechanism, is adjustable in height between a retracted state and a raised state in which the lifting plate forms a bearing for one of the end-plates. Thereby, both adaptions of width as well as of height are achieved.

### CROSS-REFERENCE TO RELATED APPLICATIONS

This application is a National Phase application under 35 U.S.C. §371 of International Application No. PCT/EP2015/075265 filed Oct. 30, 2015, which claims priority benefit to European Patent Application No. 14191306.1 filed Oct. 31, 2014, the disclosures of which are herein incorporated by reference in their entirety.

### FIELD OF THE INVENTION

The invention relates to an intervertebral fusion implant for the fusion of two adjacent vertebrae, comprising an adjustable support body, of which the lower and upper cover surfaces are designed to bear on end-plates of the adjacent vertebrae.

### BACKGROUND OF THE INVENTION

The intervertebral disks of the spinal column suffer degeneration as a result of wear or of pathological changes. If conservative treatment by medication and/or physiotherapy is ineffective, surgical treatment is sometimes indicated. In this connection, it is known for a movable or immovable implant to be inserted into the intervertebral space containing the degenerated intervertebral disk. This implant takes over the support function of the degenerated intervertebral disk and to this extent restores a stable bearing between the adjacent vertebrae. Immovable implants are also referred to as “fusion implants”.

Various surgical techniques are known for implanting the fusion implants. A traditional surgical technique involves a ventral access route, in order thereby to avoid the danger of damaging the spinal cord in the vertebral column. A ventral access route and avoidance of the spinal column also affords the opportunity to introduce a fusion implant with large cross-sectional dimensions, providing significant support for the weight-bearing vertebral bodies. However, these advantages are obtained at the price of a very long access route through the abdominal cavity or thoracic cavity of the patient, and the need to pass closely to the major blood vessels. Since complications can result, an alternative access route has become established, namely from the dorsal direction. Although the latter affords the advantage of a short route, there is the danger of collision with or damage to the spinal cord. To minimize this danger, the operation is usually performed by minimally invasive surgery. Approaches of this kind directly from the dorsal direction or more from the side are known as PLIF (posterior lumbar intervertebral fusion) or TLIF (transforaminal lumbar interbody fusion), in which the intervertebral disk is exposed from the posterior or lateral direction, respectively. Because of the small transverse incisions used in an approach by minimally invasive surgery, the size of the fusion implants is of course greatly restricted.

For treatment using the PLIF or TLIF technique, very small fusion implants are known. They afford the advantage of being able to be implanted by minimally invasive surgery thanks to their small size. However, an inherent disadvantage of their small size is that the support function is limited because of the small dimensions and is sometimes inadequate. Although a larger size of the fusion implants would improve the support function, this is impractical because of the limits of minimally invasive surgery.

It has been proposed to design an intervertebral fusion implant for the minimally invasive function in such a way that a laterally pivotable side bracket is arranged on a small, for example box-shaped support body. In a position of assembly, the side bracket is retracted into the support body. In this way, the cross section required for the implantation remains limited to that of the support body itself. At the intended site of implantation, the side bracket, which is designed as a kind of toggle expander, is pivoted out to its working position by an actuating instrument. The side bracket is thus spread open laterally. It functions with its bottom and with its top as additional support. Overall, the implant thus provides a support surface approximately in the shape of an isosceles triangle. The support surface is thus considerably increased compared to that of the support body. Against this advantage of the quite large support surface, there is the disadvantage that adaptation to the anatomical circumstances in the intervertebral space is possible only to a limited extent.

### SUMMARY OF THE INVENTION

Proceeding from the first-mentioned prior art, an object of the invention is to make available an improved intervertebral fusion implant which, while still having small dimensions, can be better adapted to different anatomical circumstances.

Solutions according to aspects of the invention lie in the features of the independent claim. Advantageous developments are the subject matter of the dependent claims.

According to one embodiment, an intervertebral fusion implant for the fusion of two adjacent vertebrae, comprising an adjustable support body, of which the lower and upper cover surfaces are designed to bear on end-plates of the adjacent vertebrae, the invention provides a side bracket, which is pivotable laterally about a hinge, and of which the bottom face and top face are designed to bear on the end-plates, and the invention further provides a lifting plate which, by means of a lifting mechanism, is adjustable in height between a retracted state and a raised state in which the lifting plate is spaced apart from the cover surface and forms a bearing for one of the end-plates.

As mentioned above, the prior intervertebral implants suffer from the disadvantage that adaptation to the anatomical circumstances in the intervertebral space is possible only to a limited extent. It also has to be noted that while the upper and lower cover surfaces of prior art implant support bodies are designed to bear on the end-plates of adjacent vertebrae, said surfaces usually have a planar surface design which cannot be tailored to the corresponding vertebra endplate not having such a planar surface but a concave surface facing the planar cover surfaces of the implant body. This disadvantage also applies to prior art intervertebral implants (see e.g. EP 2 2 777 633 A2, WO 2013/006669 A2, WO 2011/011609 A2, WO 2013/158294) having an expandable mechanism between both upper and lower surfaces allowing the adjustment of the implant between the end-plates of adjacent vertebrae in height only.

An aspect of the implant, according to some embodiments, is that it can be expanded in both directions—the lateral direction and the vertical direction. While the lateral expansion is provided by the laterally pivotable side bracket arranged on a support body which can be pivoted out to its working position by an actuating instrument, the vertical expansion is provided by the lifting plate. None of said above cited prior art documents provide said both functionalities in concert, i.e. the option of expanding the implant in the lateral and vertical direction. Having said both functionalities in concert as per the implant as defined in the claims allows for a tailored positing of the implant within the intervertebral space in order to provide the implant's support function at the best.

Furthermore, the applicant has surprisingly noted that the implant comprising an additional lifting plate allows the implant's ingrowth into the intervertebral space much faster compared to implants not having said lifting plate and provides the following explanation thereof:

Each medical implant constitutes a foreign body for the implantee and therefore brings about a complex biological interaction on a very wide variety of different levels. One of the most important reactions of the body is recruitment of osteogenic stem cells to the implant surface, known as osteoconduction. In this process, in a first step, the implant surface absorbs fibrinogen, to which there is attachment of platelets, which on their part release osteogenic growth factors when activated and induce migration of osteogenic stem cells to the implant, specifically the implant surface. The osteogenic stem cells secrete an organic bone matrix, which is mineralized by calcium phosphate deposition. In the ideal case, the implant is tightly joined to the bone following completed osteoconduction, which imparts primary stability, and osteointegration, which imparts secondary stability.

The applicant therefore assumes that implant's faster ingrowth into the intervertebral space compared to implants not having said lifting plate is triggered by the additional surface presented to the osteogenic stem cells once the implant has been implanted within the intervertebral space. It is known that the more implant surface is presented to the osteogenic stem cells the faster the implant gets fused to and integrates within the bone material of adjacent vertebrae. The fusion implant as per the present invention obviously provides via its lifting plate an additional surface said osteogenic stem cells can be adhered to, i.e. the osteoinduction and osteointegration process finds additional support by said lifting plate independent from and in addition to said process on the upper and lower support surfaces of the implant.

Furthermore, it has been surprisingly noted that the lifting plate on the cover surface of the support body reinforces and braces the implant as a whole, in particular once the lifting plate has been raised and is spaced apart from the cover surface of the support body. Due to said stiffening effect by said lifting plate more lateral forces can be compensated by the implant. Furthermore, said additional stiffening by the lifting plate opens the door for a smaller implant size, which is a further advantage, in particular if a less as possible invasive surgery becomes an issue.

In addition, said lifting plate above the cover surface of the support body additionally protects the inside of the support body from external influences, wherein said inside may house for example the lifting mechanism. Said lifting mechanism inside the support body thereby becomes less exposed to external forces or ingrowth of bone tissue. The later might become a major disadvantage, at least for the following two reasons: First, the inner compartment of the support body as well as the elements therein (e.g. a lifting mechanism) may not comprise a biocompatible surface which may trigger irritations and inflammation. Second, if the implant must be removed from the patient's body or replaced by another implant which goes in hand with adjusting the lifting plate in its retracted state prior to its removal from the intervertebral space, then this might become impossible due to damages or blocking of the mechanism. In one embodiment of the present invention the cover surface of the implant body is substantially a closed surface. Said substantially closed surface may still comprise an opening or openings for e.g. the holder(s) of the lifting plate extending through the cover surface into the implant body for connection with the lifting mechanism which might be housed in the interior space of said body.

Furthermore, if the lifting plate has a reduced dimension compared to the implant body's cover surface it is spaced apart in the raised state, then this allows a more tailored installation of the implant's surfaces to the vertebra's end plate and ideally closely follows the vertebra's surface in the attachment region. In other words, the grading from the lifting plate to the support body's surface allows much better to follow the vertebra's non-planar end-plate surface.

In addition, the lifting plate can be angled to the support body, preferably with respect to the lower cover surface of the support body. One major advantage in this respect is that the angled configuration of the lifting plate aids in restoring proper lordosis. In other words, the normal inward lordotic curvature of the lumbar and cervical regions of the spine can be restored by said angled lifting plate thereby providing the patient with a stronger back and curved structure as in healthy persons. The whole lifting mechanism may be angled and/or the lifting plate with its upper surface. In a preferred embodiment of the invention the angle between the lifting plate's surface and the support body, and in particular between the lifting plate's surface and the lower of the lifting plates cover surface is about 2° to about 15°, preferably about 3° to about 11°, further preferably about 4° to about 8°. In a preferred embodiment of the invention said angle corresponds to the normal angle between adjacent vertebrae as it can be found in healthy persons.

Aspects of the invention are based on the concept of combining, in one intervertebral fusion implant, both an adaptation of the width, by means of an expandable side bracket, and also an adaptability of the height, by means of the adjustable lifting plate. In this way, an optimal adjustability and therefore adaptability of the intervertebral fusion implant to the particular anatomical circumstances is achieved. A large number of different, conventional intervertebral fusion implants of different widths and in particular of different heights are thus replaced. However, the invention not only permits a considerable reduction in the number of different sizes and variants of intervertebral fusion implants that have to be kept in stock; in addition, the intervertebral fusion implant according to the invention is also easier to implant. Its width is minimized by virtue of the laterally pivotable side bracket, and its height is minimized by virtue of the adjustable lifting plate, and therefore, by virtue of its small overall size, it can also be easily implanted by minimally invasive approaches with a particularly small transverse incision, yet can be expanded after implantation to provide significant cross-sectional support to the adjacent vertebral bodies. The invention thus combines easy implantation with versatility and the possibility of treating different anatomical configurations.

Aspects of the invention are based on the concept of combining a laterally pivotable side bracket and a lifting plate. The expandable side bracket increases the support surface of the intervertebral fusion implant according to the invention. The lifting plate permits adaptation to different heights in the intervertebral space. In particular, it is thus also possible to perform adaptation to different lordosis angles. Whereas many different parts are conventionally required for this purpose, an adjustable intervertebral fusion implant according to the invention will in future be sufficient. In addition, by virtue of the retractable and expandable side bracket and the height-adjustable lifting plate, the invention permits easy implantation by virtue of small dimensions of the support body in its position of assembly. The expansion has the effect that the support body moves sideways in the intervertebral space, such that it lies transversely on the anterior aspect of the ring apophysis, which is particularly well suited for the transfer of loads. Therefore, quite complex and extensive defects in the area of the intervertebral disk can also be treated by minimally invasive surgery safely and in a manner that is easy for the operating surgeon.

The lifting plate is expediently designed such that, in the retracted state, it lies flush on the support body. With this flush or preferably even recessed arrangement of the lifting plate on the support body, the lifting plate merges seamlessly into the outer contour of the support body. In this way, the lifting plate does not contribute to increasing the size of the outer contour, since it does not protrude. The non-protrusion of the lifting plate also affords the in practice very significant advantage that no additional edges pointing in the direction of implantation are formed, such that the insertion of the implant to its intended site of implantation is, according to the invention, just as easy as with an implant that does not have a lifting plate.

The lifting mechanism for the lifting plate is integral in the support body and can be designed in various ways. In a preferred embodiment, a central lifting element is provided for the lifting plate, preferably combined with means for preventing rotation. The central lifting element permits a compact structure and a generally simple and therefore much more robust design of the adjustment mechanism. A high degree of reliability can thus be achieved. With the means for preventing rotation, it is also ensured that the lifting plate safely maintains its intended orientation with respect to the support body even in the expanded state. Rotations of the lifting plate, which could possibly lead to irritation in the surrounding tissue as a consequence of rotating corners or edges, are thus effectively avoided. Alternatively, however, provision can also be made that two or more lifting elements are provided for the lifting plate. This affords the advantage that the force needed for the expansion is distributed over different points. Moreover, a separate means of preventing rotation is thus superfluous, since this is already achieved from the outset by the spatially offset arrangement of the plurality of lifting elements.

To actuate the lifting mechanism, a worm gear with a worm and a worm wheel is preferably provided. Here, the worm wheel preferably sits on the lifting element. If two lifting elements are provided, each of them has a worm wheel, wherein the worm wheels preferably work in opposite directions to each other. The oppositely directed configuration of the worm wheels affords the advantage that, by means of a worm inserted centrally between them and meshing with the two oppositely directed worm wheels, it is possible to achieve a particularly compact design of the lifting mechanism, and one that is easy to actuate.

It is not essential for the worm to be designed to remain permanently on the support body. Provision can also be made that the worm is arranged on an actuating instrument which is delivered from outside (i.e. from outside the body) through an access tube. It then suffices for the worm, with the actuating instrument, to be inserted into the support body in order to actuate the lifting mechanism only when adjustment of the lifting plate is in fact intended. After the lifting mechanism has been actuated, the actuating instrument with the worm can be removed.

The side bracket is expediently designed as an expander that comprises a pivoting arm along with an expanding arm. This results in an approximately triangular construction, which permits good and reliable guiding of the outwardly pivoting arm, and the risk of jamming during the outward pivoting, which could lead to blockage of the implant, is thereby effectively countered. It is particularly expedient if the expanding arm, in its working position, is locked on the support body. Such locking ensures that the angle is maintained even if large loads occur.

The expanding arm advantageously comprises a spindle, mounted on the support body, and a spindle block, fitted onto the spindle. By turning the spindle, the spindle block is moved along the spindle, as a result of which the expanding arm is expanded from the support body. In this way, the expansion of the expanding arm can be controlled with high force transmission and fine tuning. Provision is preferably made here that, in a position of assembly, the spindle block is arranged on an end face of the support body. Thus, the spindle block forms as it were a continuation of the support body, beyond the end face of the latter. This is particularly expedient if the spindle block is shaped such that it forms a continuation, of substantially identical contour, of the support body in the position of assembly. Thus, the same cross section of access is needed as for the support body itself, i.e. the spindle block of the expanding arm does not make implantation more difficult and also does not require a larger cross section of access. It has proven particularly useful here if the spindle is articulated on an end face of the support body and, in the position of assembly, preferably protrudes in the longitudinal direction of the support body. The spindle and the spindle block are thus located on the same end face in the position of assembly. The spindle block forms a continuation of the support body, such that, as has been described above, it does not require an additional cross section of access. Since the spindle, in the position of assembly, is likewise oriented in the direction of this end face and protrudes in the longitudinal direction of the support body, it too does not jut out beyond the lateral contour of the support body. Therefore, no extension of the cross section of access is needed for the spindle either. Overall, therefore, the expanding arm designed according to the invention bears so favorably on the support body, in the position of assembly, that only the same small cross section is needed as in the case of a support body without an expanding arm. This applies in particular when the underside and/or top of the spindle block are flush with the lower and upper cover surfaces, respectively, of the support body. This results in a continuous surface without gaps. It has proven particularly useful if the underside and the top of the side bracket are flush with the bottom surface and/or cover surface of the support body. This ensures that, after expansion, the side bracket lies with its bottom and its top reliably in contact with the end-plates of the two adjacent vertebrae, specifically in the same way as the support body itself.

To promote growth of the implant according to the invention into the intervertebral space, provision can be made that cutting teeth are arranged on the bottom surface and/or cover surface of the support body and/or on the bottom or top of the side bracket. Particularly during the expansion movement, trimming of the end-plate can be obtained with the cutting teeth, as a result of which the natural growth of bone, and therefore the desired fusion of the two vertebrae, is promoted and accelerated.

In order to allow bone chips, or other material promoting the growth of bone, to be introduced into the interior of the triangle formed by the expanded arm along with the support body, the side bracket is expediently provided with an aperture. The latter is preferably dimensioned such that it comprises preferably at least half, more preferably at least two thirds, of the lateral face of the side bracket. The bone chips, or other material promoting the growth of bone, can be introduced through this aperture. The aperture can also be used independently of this in order to leave free an access to the worm gear of the lifting mechanism, such that the access to the lifting mechanism is free both in the position of assembly and also in the working position with the side bracket expanded. It has proven particularly useful to design the side bracket as a frame construction. In this way, a high degree of strength and connection stiffness of the side bracket can be combined with a large aperture for easy introduction of material that promotes bone growth and also for good access to the gear of the lifting mechanism. Corresponding recesses are preferably formed on the support body, such that the side bracket designed as a frame construction is recessed, in the retracted state, into the support body. This permits a particularly compact structure, which also has a favorable shape for implantation.

It has proven useful to choose the length of the side bracket such that it lies in a range of 0.8 to 1.1 times the length of the support body. For practical requirements, a very favourable geometry in the expanded state is obtained if the length of the support body and the position of the hinge for the side bracket are adapted to each other in such a way that, in the expanded state, the spindle protrudes at right angles from the support body, and moreover the support body, on the one hand, and the spindle without the spindle block, on the other hand, form what is approximately an isosceles triangle. It is thus possible to achieve secure support of the end-plates, particularly in the areas near the margins thereof, which are provided with a harder cortical layer. The support function is then substantially better than with conventional implants which, because of their small size, have to be positioned more to the center of the end-plates, where the load-bearing capacity of the vertebrae is much lower. It is also thereby ensured that, in the expanded state, the support body extends exactly as far forward (anteriorly) as in the position of assembly. Thus, there is no longer any danger of irritation of tissue in front of the vertebral body. At the same time, the surgeon has the assurance that the support body with its lifting plate lies in said anterior cortical area of the vertebral body particularly suitable for load transfer and does not shift in the anterior or posterior direction as a result of the expansion.

The side bracket is preferably designed such that it pivots out at least by a distance corresponding to three times the width of the support body. This provides the implant with a basic width that permits secure support even with just a single implant in an intervertebral space.

The geometry of the side bracket is such that the length ratio of spindle, on the one hand, and expanding arm, on the other hand, is chosen such that, in the expanded state (working position), the spindle protrudes approximately transversely from the support body. This ensures optimal fanning-out and therefore an optimal widening of the support basis by the triangle formed by support body, expanding arm and spindle.

The invention further comprises an instrument set for the intervertebral fusion implant. It comprises a guide tube with an insertion rod, and an actuating rod, and furthermore a coplanar sighting tube arranged at an angle on the guide tube, such that it intersects an axis of the guide tube in front of the mouth thereof. The angle between guide tube and sighting tube is chosen such that an axis of the sighting tube intersects the axis of the guide tube at a distance in front of the mouth of the guide tube corresponding to 0.5 to 1.0 times the length of the support body. This has the effect that, when the support body is mounted on the guide tube, the sighting tube is oriented approximately centrally to the lateral face of the support body, i.e. where the access to the lifting mechanism is located in preferred embodiments. In this way, the lifting mechanism can be actuated by a worm spindle guided through the sighting tube. The angled arrangement of the sighting tube on the guide tube affords the advantage that a bilateral access to the intervertebral fusion implant according to the invention can be achieved, wherein the guide tube is guided on one side around the nerve tract in the vertebral column and the sighting tube is guided on the other side of the nerve tract. The sensitive nerve tract of the vertebral column is as it were enclosed by both tubes, and it is thus ensured that the actuating rod and/or the worm spindle cannot adversely affect the nerve cord. With the sighting tube arranged at an angle, the operating surgeon can move the worm spindle safely and with precision to the intended site of insertion in the lifting mechanism, without running the risk of damaging the nerve tract.

The insertion rod is pushed into the guide tube and connected to the intervertebral fusion implant at the posterior face thereof. To hold the implant on the guide tube for rotation therewith, the guide tube preferably has a tongue, which is designed to engage in a corresponding recess of the support body of the intervertebral fusion implant. Finally, the actuating rod is inserted through the guide tube with the insertion rod. It serves to actuate the expanding arm. It preferably itself carries a part of the actuating device, namely in the form of a thread at its front end, which thread functions as a spindle for the actuating device.

The invention further comprises a set or kit of parts comprising the intervertebral fusion implant of the present invention and the instrument set for the intervertebral fusion implant as outlined above.

### BRIEF DESCRIPTION OF THE DRAWINGS

The invention is explained in more detail below with reference to the attached drawing in which advantageous illustrative embodiments are depicted. In the drawing:

FIG. 1 shows a schematic view of an intervertebral fusion implant according to the invention in the intervertebral space between vertebral bodies;

FIG. 2 shows plan views and a partially perspective representation of the intervertebral fusion implant in the position of assembly, in the working position, and in an intermediate position;

FIG. 3 shows an exploded view of the intervertebral fusion implant with inserted worm gear;

FIG. 4 shows a view of an instrument set; and

FIG. 5 shows a detail of the attachment of an insertion instrument to the intervertebral fusion implant.

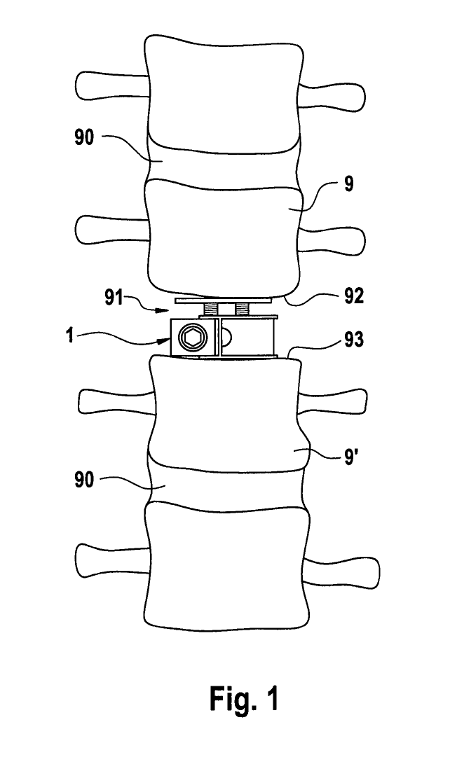

### DETAILED DESCRIPTION OF THE INVENTION

An illustrative embodiment of an intervertebral fusion implant according to the invention, designated in its entirety by reference number 1, is provided for implantation in an intervertebral space 91 between two immediately adjacent vertebral bodies 9, 9′. In a physiologically intact vertebral column, an intervertebral disk 90 is located in the intervertebral space between the vertebrae. This intervertebral disk 90 may undergo degeneration as a result of disease or wear, with the result that it has to be at least partially resected. In order to achieve sufficient support of the intervertebral space 91, despite the loss of intervertebral disk material, and to thereby prevent collapse of the vertebral column, the intervertebral fusion implant 1 is inserted into the intervertebral space 91. It provides a supporting action and thus facilitates fusion of the adjacent vertebrae 9, 9′ in a natural way through bone growth.

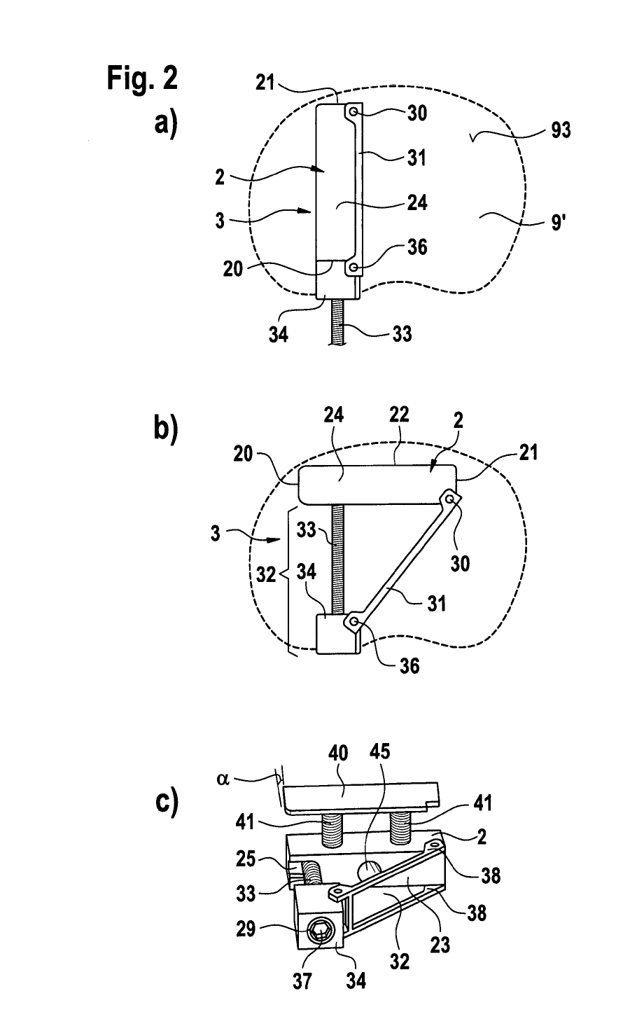

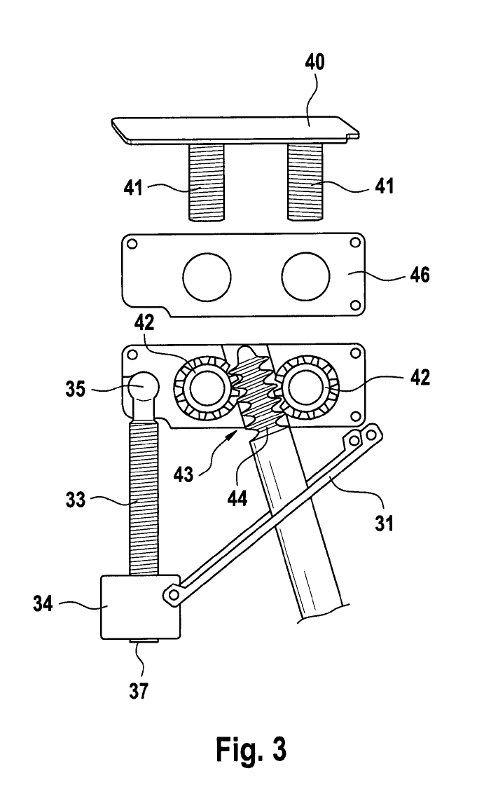

In the following explanation of the structure and function of the illustrative embodiment of the intervertebral fusion implant 1, reference is made to FIGS. 2 and 3. The intervertebral fusion implant comprises a support body 2, with a side bracket 3 which is arranged pivotably thereon via a hinge 30. The side bracket 3 is formed in two pieces, with a pivoting arm 31, which has the hinge 30 at one end thereof, and an expanding arm 32, which is connected in an articulated manner to the other end of the pivoting arm 31 via a second hinge 36. The expanding arm 32 comprises a spindle 33 and a spindle block 34. The spindle block 34 is cuboid and has the hinge 36 on its edge facing toward the support body 2.

At one end, the spindle 33 has a spherical bearing foot 35 and, at its other end, it has an actuating head 37, which is preferably designed as a hexagon socket. The spherical bearing foot 35 is mounted in a corresponding recess 25 on the end face 20 of the support body 2 and thus forms a pivot joint, wherein the recess 25 extends into the adjacent area of the lateral surface 23 of the support body 2 facing toward the expanding arm 3. The bearing foot 35 thus forms a pivot bearing, as a result of which the support body 2, which in the position of assembly bears with its end face 20 on the spindle block 34, is transferred in an arc shape, by expansion of the expanding arm 3, to its transversely oriented working position (cf. FIGS. 2a and 2b). In the position of assembly, the spindle block 34 lies flush on the end face 20 of the support body 2 and forms a continuation, of identical contour, of the cross section of the support body 2. The underside and top of the spindle block 34 are flush here with the cover and bottom of the support body 2. Moreover, the lateral face of the spindle block 34 directed away from the expanding arm 3 is flush with the corresponding opposite lateral face 22 of the support body 2. In the position of assembly, the spindle 33 lies in the longitudinal direction of the support body 2, the longitudinal direction being defined by the main axis of the support body 2 connecting the end faces 20, 21.

The expanding arm 31 of the side bracket 3 is designed as a frame construction. At the top and bottom it has a respective main strut 38, which are connected in the area of the hinge 36 by a transverse strut 39. They delimit a large central aperture 39. In the position of assembly (see FIG. 2a), the expanding arm 31 lies recessed on the support body 2, wherein the longitudinal supports 38 engage in recesses 28 formed on the top and bottom edge of the lateral face 23.

By actuation of the spindle 33, by means of turning the spindle using an actuating tool which engages on the spindle head 37, the spindle block 34 and the support body 2 are moved away from each other, as a result of which the pivoting arm 31 is released and pivots away from its position of assembly directly on the lateral face 23 of the support body 2, and at the same time the support body 2 is pivoted into a direction transverse to the spindle 33 via the pivot hinge formed by the spindle foot 35. Since the spindle block 34 is fixed by the actuating tool, it remains stationary.

A lifting plate 40 is also provided, which forms a cover 24 of the support body 2. The lifting plate 40 is actuated by a lifting mechanism 4, which has two lifting spindles 41, and two worm wheels 42 which are fitted on the lifting spindles 41 and which, on their inside, have a mating thread for screwing onto the lifting spindle 41 and, on their outside, have teeth for cooperating with a worm spindle 44. The worm wheels 42 and the worm spindle 44 form a worm gear 43, which is accessible from outside via an actuation opening 45 in the lateral face 23 of the support body 2. The worm gear 43 is arranged in the interior of the support body 2 and is provided with a covering 46. The worm gear 43 functions such that an actuating instrument with a worm spindle 44 at its front end is pushed through the aperture 39 in the expanding arm 31 and through the access opening 45 in the lateral face 23 into the interior of the support body 2, where the worm spindle 44 comes to rest centrally between the two worm wheels 42 and meshes with these. By turning the actuating instrument with the worm spindle 44, the worm wheels 42 are brought into opposing rotation, as a result of which the two lifting spindles 41 are moved upward and thus raise the lifting plate 40. The lifting plate 40 thus reaches a position spaced apart from the support body 2, the size of the space being defined by the number of revolutions of the worm spindle 44. In this way, a stepless regulation of the height of the lifting plate 40 can take place. Said lifting plate 40 can be angled to the support body 2 as indicated by the angle “α”. Said angle α can be about 2° to about 15°, preferably about 3° to about 11°, further preferably about 4° to about 8°. Said angle may correspond to the normal angle between adjacent vertebrae as it can be found in healthy persons at the intervertebral fusion site.

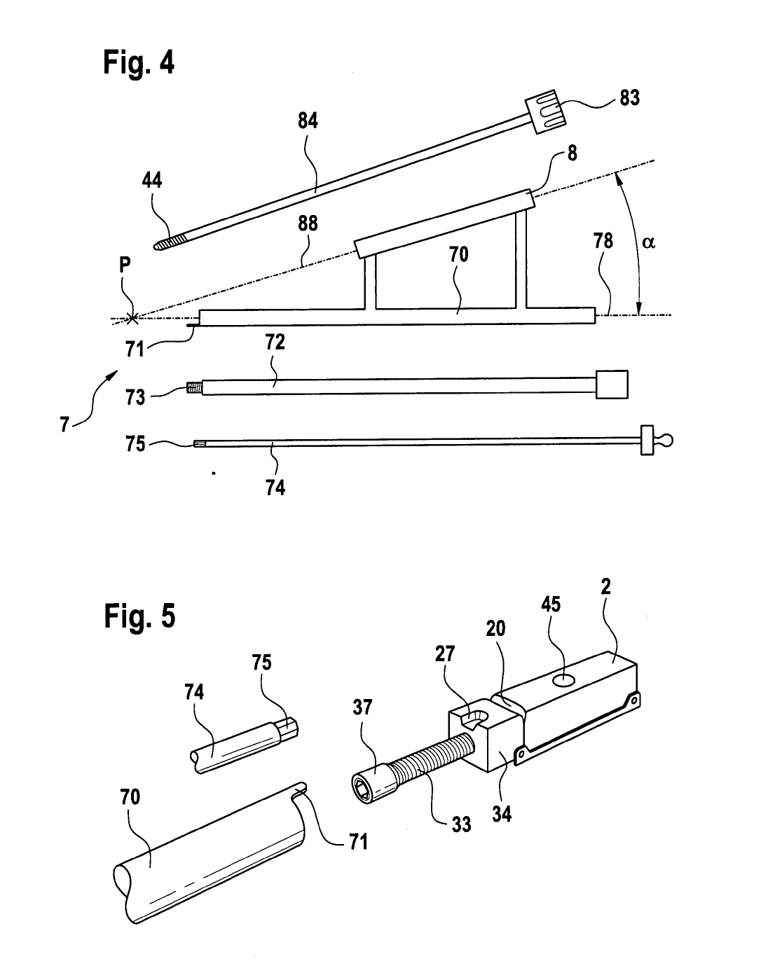

To insert and actuate the intervertebral fusion implant, an instrument set is provided, as is shown in FIG. 4. It comprises a guide tube 70 with a tongue 71 at its front end, which tongue 71 is designed to engage in a correspondingly shaped and complementary recess 27 on the spindle block 34 bearing on the end face 20 of the support body 2. By means of this form-fit connection, the support body 2 with the spindle block 34 is arranged on the guide tube 70 so as to rotate therewith. In order to fix the support body 2 with the spindle block 34 on the guide tube 70, an insertion rod 72 is provided which has a thread 73 at its front end. This thread 73 engages in a retaining thread 29, which is designed as an internal thread on the end face of the spindle block 34 directed away from the support body. By turning the insertion rod 72, a tension-resistant screw connection to the spindle block 34 and to the support body 2 is achieved.

A sighting tube 8 is arranged fixedly and in a coplanar manner on the guide tube 70. It is oriented at an angle α to the axis 78 of the guide tube, specifically in such a way that the axis 88 of the sighting tube 8 intersects the axis 78 of the guide tube 70 at a point p, which is located in front of the mouth of the guide tube 70 by a distance corresponding approximately to half the length of the support body 2. The sighting tube 8 serves to receive a rotation rod 84 which, at its rear end, has a grip 83 for rotating it and, at its front end, has the worm spindle 44. Moreover, the instrument set comprises an actuating rod 74 which, at its front end, has a hexagon 75 matching the head 37 of the spindle 33.

For the implantation, the insertion rod 72 is pushed through the guide tube 70, and the insertion rod 72, with the thread 73 at its front end, receives the support body 2 and holds the latter on the front end of the guide tube 70. Securing against rotation is additionally achieved by the tongue 71 at the front end of the guide tube 70 engaging in the recess 27 on the support body 2. By means of minimally invasive surgery, the intervertebral fusion implant 1, thus mounted on the guide tube 70 rigidly and in a manner secure against rotation, can then be introduced by the chosen access route (for example by the PLIF operating technique) to the intended site of implantation in the intervertebral space 91, until it has reached the intended site of implantation in which the anterior end face 21 of the support body 2 lies flush with the anterior margin of the cover surface 93 of the vertebra (see FIG. 2a). When the implant 1 is located at the intended site of implantation, the expanding arm 3 is expanded in a first step. For this purpose, the actuating rod 74 is pushed through the insertion rod 72, which is hollow for this purpose, until the hexagon 75 at the front end engages in the hexagon socket on the head 37 of the spindle 33. By turning the actuating rod 74, the spindle 33 is rotated, and the support body 2 is moved away from the spindle block 34 in the manner described below and the pivoting arm 31 is spread open. The spindle bock 34 remains stationary on account of its being fixed on the guide tube 70. The chosen length ratio of expanding arm 3 to support body 2 ensures that the support body 2 in the expanded state protrudes in the anterior direction (see FIG. 2b) exactly as far as in the position of assembly (see FIG. 2a). After the expansion has been completed, the actuating rod 74 can be removed. The rotation rod 84 is now pushed through the sighting tube 8. It will be noted here that, by virtue of the angle α, the sighting tube 8 targets the implantation site from the contralateral side. That is to say, if the guide tube 70 is guided to the left along the nerve tract in the vertebral column, the sighting tube 8 is located to the right. The fixed angle ensures that the sighting tube 8, with its rotation rod 84 pushed through it, maintains a sufficient distance from the sensitive nerve tract in the vertebral column. In this way, the worm spindle 44 on the rotation rod 84 can be safely pushed through the sighting tube 8 until it finally enters the support body 2 of the intervertebral fusion implant. By virtue of the angle α on the sighting tube 8, the worm spindle 44 of the rotation rod 84 passes through the aperture 39 of the pivoting arm 31 into the access opening 45 in the lateral face 23 of the support body 2 and thus comes to bear in its intended position centrally between the two worm wheels 42. By turning the rotation rod 84 by means of the grip 83, the worm spindle 44 is actuated, as a result of which the worm wheels 42 rotate and the lifting plate 40 is elevated above the lifting spindles 41. Once the lifting plate 40 has reached the desired position, the rotation rod 84 can be removed.

1. An intervertebral fusion implant for the fusion of two adjacent vertebrae, comprising:
an elongate, adjustable support body comprising lower and upper cover surfaces that are configured to bear on end-plates of the adjacent vertebrae;
a side bracket that is pivotable laterally about a hinge, wherein a bottom face and a top face of the side bracket are configured to bear on the end-plates; and
a lifting plate located on the lower or upper cover surface of the support body, the lifting plate being adjustable in height by a lifting mechanism between a retracted state and a raised state in which the lifting plate is spaced apart from the lower or upper cover surface and forms a bearing for one of the end-plates.

2. The intervertebral fusion implant of claim 1, wherein the lifting plate, in the retracted state, lies flush on the support body.

3. The intervertebral fusion implant of claim 1, wherein a central lifting element is provided for the lifting plate.

4. The intervertebral fusion implant of claim 1, wherein two or more lifting elements are provided for the lifting plate.

5. The intervertebral fusion implant of claim 1, wherein the lifting mechanism comprises a worm gear with worm and worm wheel.

6. The intervertebral fusion implant of claim 4, wherein the two or more lifting elements comprise worm wheels that work in opposite directions to each other.

7. The intervertebral fusion implant of claim 5, wherein the worm is configured to be insertable and removable.

8. The intervertebral fusion implant of claim 7, wherein the worm sits at the tip of an actuating instrument that is configured to be delivered from outside through an access tube.

9. The intervertebral fusion implant of claim 1, wherein the side bracket is configured as an expander with a pivoting arm and an expanding arm.

10. The intervertebral fusion implant of claim 9, wherein the expanding arm, in a working position, is locked on the support body.

11. The intervertebral fusion implant of claim 9, wherein the expanding arm comprises a spindle that is mounted on the support body, and a spindle block that is fitted onto the spindle.

12. The intervertebral fusion implant of claim 11, wherein, in a position of assembly, the spindle block is arranged on an end face of the support body.

13. The intervertebral fusion implant of claim 11, wherein the spindle is articulated on an end face of the support body.

14. The intervertebral fusion implant of claim 11, wherein an underside and a top of at least one of the pivoting arm and the spindle block are flush with the lower and upper cover surfaces, respectively, of the support body.

15. The intervertebral fusion implant of claim 9, wherein the side bracket has an aperture that comprises at least half of a lateral face of the side bracket.

16. The intervertebral fusion implant of claim 15, wherein the aperture leaves free an access to the worm gear at least in the expanded state of the side bracket.

17. The intervertebral fusion implant of claim 9, wherein the side bracket is configured as a frame construction, and the support body is formed with corresponding recesses into which the side bracket fits in the retracted state.

18. The intervertebral fusion implant of claim 1, wherein the support body and the side bracket form a right-angled triangle in the expanded state.

19. The intervertebral fusion implant of claim 18, wherein a length of the support body and a length of a hinge for the side bracket are configured such the support body and the hinge form an isosceles triangle in the expanded state.

20. The intervertebral fusion implant of claim 18, wherein the side bracket has a length of approximately 0.8 to 1.1 times a length of the support body.

21. An instrument set for an intervertebral fusion implant that comprises an elongate, adjustable support body comprising lower and upper cover surfaces that are configured to bear on end-plates of adjacent vertebrae, a side bracket that is pivotable laterally about a hinge, wherein a bottom face and a top face of the side bracket are configured to bear on the end-plates, and a lifting plate located on the lower or upper cover surface of the support body, the lifting plate being adjustable in height by a lifting mechanism between a retracted state and a raised state in which the lifting plate is spaced apart from the lower or upper cover surface and forms a bearing for one of the end-plates, wherein the instrument set comprises:
a guide tube, an actuating rod, and a coplanar sighting tube arranged at an angle on the guide tube such that the sighting tube intersects an axis of the guide tube in front of a mouth of the guide tube.

22. The instrument set as claimed in claim 21, wherein a tongue for engaging in the support body of the intervertebral fusion implant is arranged on the guide tube at the front end.

23. The instrument set of claim 21, wherein the actuating rod has a thread at its front end, the thread being configured as a spindle for an actuating device of the side bracket.

24. The instrument set of claim 21, further comprising a worm spindle for actuating the lifting mechanism, wherein the sighting tube is configured to receive the worm spindle.

25. The intervertebral fusion implant of claim 3, wherein the implant is configured to prevent the central lifting element from rotating.

26. The intervertebral fusion implant of claim 5, wherein and the worm wheel sits on the lifting element.

27. The intervertebral fusion implant of claim 7, wherein the worm is configured to be inserted centrally between two lifting elements that re provided for the lifting plate.

28. The intervertebral fusion implant of claim 12, wherein the spindle block forms a continuation of a contour of the support body.

29. The intervertebral fusion implant of claim 13, wherein, in the position of assembly, the spindle protrudes in the longitudinal direction of the support body.

30. The intervertebral fusion implant of claim 15, wherein the aperture comprises at least two thirds of the lateral face of the side bracket.

31. The intervertebral fusion implant of claim 16, wherein the aperture leaves free an access to the worm gear also in the retracted state.

32. The instrument set of claim 21, wherein the sighting tube intersects an axis of the guide tube in front of a mouth of the guide tube at a distance corresponding to 0.5 to 1.0 times a length of the support body.
